(19) United States
(12) Patent Application Publication
(10) Pub. No.: US 2019/0090853 A1
(43) Pub. Date: Mar. 28, 2019

(54) INFORMATION PROCESSING APPARATUS AND INFORMATION PROCESSING METHOD

(71) Applicant: CANON KABUSHIKI KAISHA, Tokyo (JP)

(72) Inventors: Fumitaro Masaki, Brookline, MA (US); Kouichi Kato, Yokohama-shi (JP)

(21) Appl. No.: 16/130,422

(22) Filed: Sep. 13, 2018

(30) Foreign Application Priority Data
Sep. 19, 2017 (JP) 2017-178749

(51) Int. Cl. A61B 8/08; A61B 5/00; A61B 8/14

(52) U.S. Cl. CPC A61B 8/5207; A61B 5/0095; A61B 8/085; A61B 8/0891; A61B 8/14

(57) ABSTRACT

Provided is an information processing apparatus, including: an image acquiring unit configured to acquire a photoacoustic image which originates from a photoacoustic wave generated by irradiating an object with light; an interpretation result acquiring unit configured to acquire interpretation result information, which represents a result of interpreting the photoacoustic image; a biopsy result acquiring unit configured to acquire biopsy result information, which represents a result of biopsy performed on the object; and a data generating unit configured to generate data in which the photoacoustic image, the interpretation result information and the biopsy result information are associated one another.

### BACKGROUND OF THE INVENTION

### Field of the Invention

The present invention relates to an information processing apparatus and an information processing method.

### Description of the Related Art

Research on optical imaging, which acquires characteristic information on an object by irradiating the object with light, is ongoing. In particular, a technique called “photoacoustic tomography” (PAT) is receiving attention. Photoacoustic tomography is a technique to detect an acoustic wave (photoacoustic wave), which is generated from an object due to the photoacoustic effect when the object is irradiated with light, and to acquire the characteristic information inside the object. A photoacoustic tomography apparatus detects the photoacoustic wave which is generated and propagated inside the object, and analyzes the acquired signals, so as to image the optical characteristic values, particularly absorption coefficient distribution, inside the object.

The initial sound pressure of the photoacoustic wave, which is generated from a region of interest inside the object, is determined based on: the Gruneisen coefficient which is approximately constant for each object; the absorption coefficient of the light absorber in the region of interest; and the light quantity in the region of interest. Therefore the absorption coefficient distribution inside the object can be acquired if the initial sound pressure distribution is determined by a known image reconstruction method using the intensity of the photoacoustic wave detected by an acoustic wave detector, and the light quantity distribution is determined based on the irradiated light quantity. This means that the blood vessel distribution inside the object can be imaged by acquiring the absorption coefficient distribution of hemoglobin which is contained in blood at high concentration.

It is also expected to image the oxygen saturation distribution inside the object by performing the photoacoustic measurement using lights having two wavelengths, of which absorption characteristics with respect to oxyhemoglobin and deoxyhemoglobin are different respectively. Here it is assumed that many new blood vessels are formed around a tumor tissue, such as a breast cancer, in order to receive oxygen and nutrition. Based on this assumption, it is under consideration that diagnosis for a tumor is performed based on photoacoustic images acquired by the photoacoustic measurement.

US Patent Application Publication No. 2016/0343132 discloses a method of specifying a position of a breast tumor using an ultrasound apparatus, then determines whether the tumor is benign or malignant based on the form, number, density and flow state of blood vessels, the amount of hemoglobin, oxygen saturation degree and the like acquired by the photoacoustic measurement.

In the case of estimating a benign or malignant tumor, of which position is specified by the ultrasound apparatus, based on the blood vessel information in the photoacoustic image, as disclosed in US Patent Application Publication No. 2016/0343132, the photoacoustic image is first interpreted by an interpreter. If malignancy is suspected as a result of the interpretation, a biopsy is performed, and it is determined whether the tumor is benign or malignant.
Patent Literature 1: US Patent Application Publication No. 2016/0343132

### SUMMARY OF THE INVENTION

However, interpretation is based on subjective evaluation, and the quality thereof depends on the interpreter. A correct/incorrect of the interpretation is decided by performing a biopsy, which is a definitive diagnosis, but the acquired information cannot be fully utilized without database that associates the interpretation result and the biopsy result.

With the foregoing in view, it is an object of the present invention to provide a method of constructing a database that is effective for interpreting photoacoustic images.

The present invention provides an information processing apparatus, comprising:

an image acquiring unit configured to acquire a photoacoustic image which originates from a photoacoustic wave generated by irradiating an object with light;

an interpretation result acquiring unit configured to acquire interpretation result information, which represents a result of interpreting the photoacoustic image;

a biopsy result acquiring unit configured to acquire biopsy result information, which represents a result of biopsy performed on the object; and

a data generating unit configured to generate data in which the photoacoustic image, the interpretation result information and the biopsy result information are associated with one another.

The present invention also provides an information processing method, comprising:

an image acquiring step of acquiring a photoacoustic image which originates from a photoacoustic wave generated by irradiating an object with light;

an interpretation result acquiring step of acquiring interpretation result information, which represents a result of interpreting the photoacoustic image;

a biopsy result acquiring step of acquiring biopsy result information, which represents a result of biopsy performed on the object; and

a data generating step of generating data in which the photoacoustic image, the interpretation result information, and the biopsy result information are associated with one another.

The present invention also provides a non-transitory computer readable storage medium storing a program causing a computer to execute an information processing method, comprising:

an image acquiring step of acquiring a photoacoustic image which originates from a photoacoustic wave generated by irradiating an object with light;

an interpretation result acquiring step of acquiring interpretation result information, which represents a result of interpreting the photoacoustic image;

a biopsy result acquiring step of acquiring biopsy result information, which represents a result of biopsy performed on the object; and

a data generating step of generating data in which the photoacoustic image, the interpretation result information, and the biopsy result information are associated with one another.

According to the present invention, a method of constructing a database that is effective for interpreting photoacoustic images is provided.

Further features of the present invention will become apparent from the following description of exemplary embodiments with reference to the attached drawings.

### BRIEF DESCRIPTION OF THE DRAWINGS

FIG. 1 is a block diagram depicting an apparatus configuration according to Example 1;

FIG. 2 is a block diagram depicting a server configuration according to Example 1;

FIG. 3 is a flow chart depicting processing according to Example 1;

FIG. 4 is a flow chart depicting processing according to Example 2;

FIG. 5 is a flow chart depicting processing according to Example 3; and

FIG. 6 is a diagram depicting the number of blood vessels and the number of cases according to Example 3.

### DESCRIPTION OF THE EMBODIMENTS

Preferred embodiments of the present invention will be described with reference to the drawings. The dimensions, materials and shapes of the components and the relative positions thereof, which will be described below, may be appropriately changed depending on the configuration and various conditions of an apparatus to which the invention is applied. Therefore, the following description is not intended to limit the scope of the invention.

The present invention relates to a technique to detect an acoustic wave propagated from an object, generate characteristic information (object information) inside the object, and acquire this information. This means that the present invention may be regarded as an acoustic apparatus or a control method thereof, or an object information acquiring apparatus or a control method thereof. The present invention may also be regarded as an object information acquiring method or a signal processing method.

The present invention may also be regarded as an information processing apparatus or an information processing method. The present invention may also be regarded as an image information processing apparatus or an image information processing method, since image information inside the object in particular, among information processing, is processed. Furthermore, the present invention may be regarded as a program which causes an information processing apparatus equipped with such hardware resources as a CPU and memory to execute these methods, or a non-transitory computer readable storage medium storing the program.

The object information acquiring apparatus of the present invention includes a photoacoustic apparatus utilizing the photoacoustic effect, which irradiates an object with light (electromagnetic wave), receives an acoustic wave generated thereby inside the object, and acquires the characteristic information of the object as the image data. In this case, the characteristic information is information on characteristic values corresponding to a plurality of positions inside the object, which are generated using signals originating from the received photoacoustic wave.

The characteristic information acquired by the photoacoustic apparatus indicates a generation source distribution of an acoustic wave which was generated by light irradiation, an initial sound pressure distribution inside the object, a light energy absorption density distribution and absorption coefficient distribution which are derived from the initial sound pressure distribution, and a concentration distribution of a substance constituting a tissue. The concentration distribution of a substance is, for example, an oxygen saturation distribution, a total hemoglobin concentration distribution, and an oxy/deoxyhemoglobin concentration distribution.

The characteristic information, which is object information at a plurality of positions, may be acquired as a two-dimensional or three-dimensional characteristic distribution. The characteristic distribution may be generated as image data representing the characteristic information inside the object. The image data may be generated as three-dimensional volume data by image reconstruction, for example.

The acoustic wave referred to in the present invention is typically an ultrasound wave, and includes an elastic wave called a “sound wave” and an “acoustic wave”. A signal (e.g. electric signal) converted from an acoustic wave by a transducer or the like is called an “acoustic signal” or a “reception signal”. The ultrasound wave or acoustic wave referred to in this description is not intended to limit the wavelength of the elastic wave. An acoustic wave generated by the photoacoustic effect is called a “photoacoustic wave” or a “light-induced ultrasound wave”. A signal (e.g. electric signal) originating from a photoacoustic wave is also called a “photoacoustic signal”. An image generated from a photoacoustic signal by image reconstruction or the like is called a “photoacoustic image”.

Among the various modalities to diagnose a living body by images, the history of photoacoustic tomography is relatively new, hence basic data thereof is limited. Further, the interpretation techniques are not fixed, and the accuracy of interpretation greatly depends on the experience and skill of the individual interpreter. Therefore, it is expected to integrate and create a database of interpreter' knowledge for the skill development and for the education of interpreters to interpret images. This database is, for example, a database of photoacoustic images associated with interpretation results and biopsy results (if performed). In the case when a biopsy was performed and the biopsy result (definitive result) is acquired, it is preferable that the interpretation result and biopsy result are corresponded in the created data, then the skills, habits and tendencies of interpreters may be analyzed or the characteristics of the images and biopsy results may be compared. Further, the interpretation result may be corrected based on the tendencies of the interpretation, or this information may be used for the training of interpreters.

### Example 1

### Basic Configuration

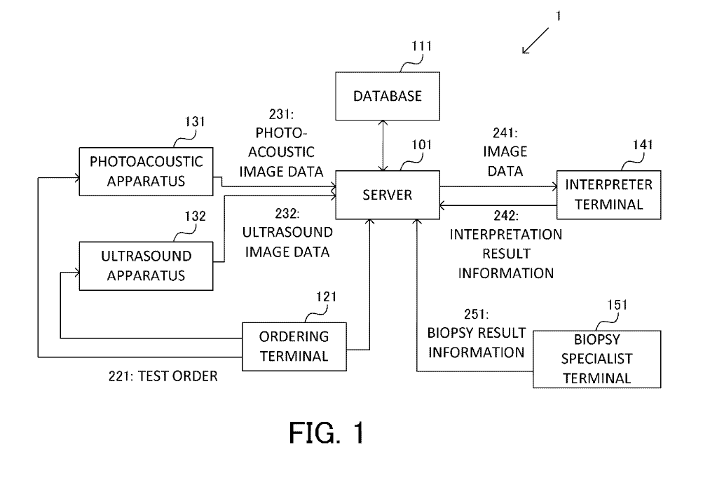

FIG. 1 indicates a general configuration of an information processing apparatus 1 according to Example 1. A server 101 processes image data acquired by each modality. A database 111 stores the image data received from the server 101, and outputs the image data upon request.

An ordering terminal 121 is a terminal for an examiner (e.g. physician) who diagnosed an examinee (patient) to order an inspection with each modality. The modality in Example 1 is assumed to be photoacoustic tomography and ultrasound echo measurement. A photoacoustic apparatus 131 performs photoacoustic tomography measurement for an object, which is a part of the examinee, in accordance with the operation of the examiner, and sends photoacoustic image data 231 to the server 101. An ultrasound apparatus 132 performs ultrasound echo measurement on the object in accordance with the operation of the examiner, and sends ultrasound image data 232 to the server 101.

An interpreter terminal 141 is a terminal for an interpreter (e.g. radiologist) to interpret the image of the object (photoacoustic image or ultrasound image). An image based on image data 241, which was read from the database 111 via the server 101, is displayed on the interpreter terminal 141. Interpretation result information 242, such as the comments of the interpreter, is sent to the server 101.

If it is determined that a biopsy is necessary as a result of the interpretation, a biopsy is ordered to a biopsy specialist. The biopsy specialist inputs biopsy result information 251, and sends it to the server 101 via a biopsy specialist terminal 151. A biopsy specialist is, for example, a physician who samples the tissue, a technician who performs the biopsy, a pathologist who attaches a comment or the like.

Thereby imaging using a modality, which the examiner determined as necessary, is performed, and the imaging result is stored in the database 111 along with the comments of the interpreter and the biopsy result.

The above configuration is one application example. CT, MRI, X-ray apparatus or the like may be used for the modality. The examiner, the interpreter and the biopsy specialist may be different individuals or a same individual. On the other hand, each terminal for ordering, interpretation and biopsy may be a same terminal.

And the location of each terminal for ordering, interpretation and biopsy may be distributed. Further, all the information processing operations need not be performed by the server 101, or all data need not be integrated in the database 111. The servers and databases may be distributed for each function or for each location.

Concrete Configuration

The server 101 is typically a DICOM server, which manages and transmits/receives in DICOM (Digital Imaging and Communication in Medicine) format. The server 101 includes a function to receive the photoacoustic image data 231 and the ultrasound image data 232, stores this data in the database 111, and includes a function to link or integrate each image data with the interpretation result information 242 and the biopsy result information 251. The data stored in the database 111 is not limited to DICOM format, but may be based on another data standard or on a format unique to an apparatus.

The possible data that may be stored in the database 111 is patient information on the characteristics of a patient, inspection information on an inspection, image information on image data, interpreter information on an interpreter, interpretation result information and biopsy result information.

Patient information is, for example, such patient information as name, age, gender, medical history and the like. Inspection information is, for example, modality, date, apparatus information, apparatus operation parameters and the like. Image information is, for example, image size and number of bits.

Interpreter information is, for example, information specifying an interpreter (e.g. name), tendencies of past interpretations, skill evaluating, result of skill evaluating by others and the like.

Interpretation result information is, for example, an opinion whether a tumor is benign or malignant, category classification, necessity for biopsy and the like. More detailed information, such as a number and density of blood vessels existing in a certain region of the image, and oxygen saturation degree of this region, are also included in the interpretation result information. The time required for an interpreter to interpret data may be included in either the interpreter information or interpretation result information.

Biopsy result information is, for example, information on a biopsy specialist, and information on whether the tumor is benign or malignant.

For the server 101, an information processing apparatus (e.g. workstation, PC), which includes a CPU, memory, input/output unit and the like, is preferable. By installing a program that processes data in DICOM format, this information processing apparatus can function as the server 101. For the database 111, a storage device (e.g. HDD, SSD) that is controlled by a database management system, is preferable. The functional blocks that perform processing related to control of the database, out of the functional block group that operates on the server, correspond to the data generating unit of the present invention. The data generating unit of the present invention may further include the database itself.

The ordering terminal 121 is typically an information processing terminal constituting an ordering system. An information processing apparatus similar to the server 101 may be used, or a thin client, which has only a function to present information to the examiner and a function to receive input from the examiner, may be used.

The photoacoustic apparatus 131 includes: a light source which generates light, an optical system which guides the light to the object, a probe which receives a photoacoustic wave generated from the object and converts the photoacoustic wave into a photoacoustic signal, and a signal processing unit which processes and reconstructs the photoacoustic signal to generate the photoacoustic image data 231.

For the light source, a laser, a light emitting diode, a flash lamp or the like may be used. If a wavelength variable light source is used, the oxygen saturation degree and substance concentration inside the object can be used. For the optical system, such optical elements as an optical fiber, a mirror, a prism, a lens and the like may be used. The probe includes an element that converts an acoustic wave into an electric signal. For the element, a piezoelectric element, a capacitance type element, a Fabry-Perot element and the like may be used. If a plurality of elements are disposed linearly or in an array in the probe, the measurement speed and image accuracy improve.

The probe may be a hand held type. A scanning mechanism, to mechanically change the relative positions of the probe and object, may be disposed. The signal processing unit includes: an AD converter which converts an electric signal into a digital signal, an amplifier which amplifies an electric signal, and an information processing circuit which executes a known image reconstruction method. The server 101, instead of the photoacoustic apparatus 131, may perform the image reconstruction processing. In this case, the server 101 receives the digital photoacoustic signal from the photoacoustic apparatus 131.

The ultrasound apparatus 132 includes a probe and a signal processing unit. The probe transmits an ultrasound wave to an object, then receives an ultrasound echo reflected from the object, and converts the ultrasound echo into an ultrasound signal. The signal processing unit processes the ultrasound signal, and reconstructs the data to generate ultrasound image data 232, which reflects the difference of acoustic impedance of the tissue inside the object.

For the probe, elements similar to those of the photoacoustic apparatus may be used. A same probe may be used for both transmission and reception of the ultrasound wave, or a probe for transmission and a probe for reception may be disposed separately. For the signal processing unit as well, a configuration similar to the photoacoustic apparatus may be used. Instead of disposing the photoacoustic apparatus and the ultrasound apparatus separately, a same apparatus may perform both the photoacoustic tomography and the ultrasound echo measurement.

The interpreter terminal 141 is an information processing terminal used by the interpreter. A terminal implemented by installing a DICOM viewer program in an information processing apparatus, similar to the server 101, may be used, or a thin client terminal, which has only a function to present information to the interpreter and a function to receive input from the interpreter, may be used. In Example 1, the interpretation is performed based on visual confirmation, and the interpreter manually inputs the interpretation result information via the terminal. However, for the interpreter, artificial intelligence (AI)-based software may be used instead of a human interpreter. In this case, the interpretation software may be installed on the server 101.

The biopsy specialist terminal 151 is an information processing terminal which the biopsy specialist uses to input the biopsy result. For the biopsy specialist terminal 151 as well, an information processing apparatus similar to the server 101 or a thin client terminal may be used. Further, instead of manually inputting the biopsy result information, a biopsy apparatus, which analyzed a sample acquired from the examinee via a needle or the like, may directly send the biopsy result to the server 101.

Functional Blocks of Server

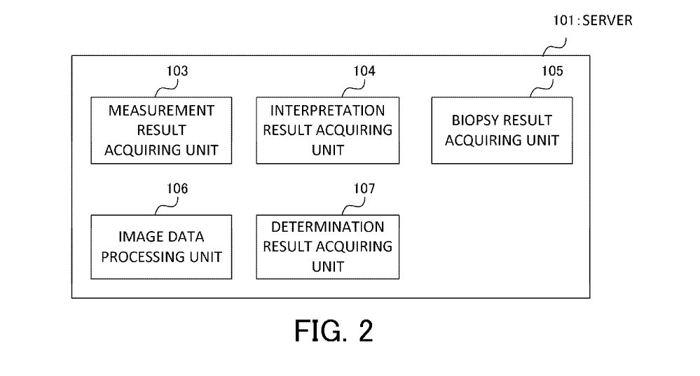

FIG. 2 is a functional block diagram of the server 101. In FIG. 2, each function performed by the server is indicated by a block. Each block may not always have a physical configuration (e.g. a circuit), but may be implemented virtually as a program module. Conversely, a part of these composing elements (e.g. a signal processing unit of the photoacoustic apparatus or the ultrasound apparatus, a server function portion which a thin client used as a terminal) may be included in the server 101. The server, database and respective terminals may be collectively regarded as the information processing apparatus of Example 1, or the server portion alone may be regarded as the information processing apparatus.

The measurement result acquiring unit 103 receives the photoacoustic image data 231 and the ultrasound image data 232 from the measurement apparatus. The interpretation result acquiring unit 104 receives the interpretation result information 242 from the interpreter terminal 141. The biopsy result acquiring unit 105 receives the biopsy result information 251 from the biopsy specialist terminal 151. The image data processing unit 106 performs various data processing operations (e.g. conversion to a predetermined format, correction of image to be appropriate for interpretation) for the image data when necessary. A determination result acquiring unit 107 performs the later mentioned determining processing in Example 1. The server may also include an interpreter information acquiring unit which acquires the interpreter information. The measurement result acquiring unit corresponds to the image acquiring unit of the present invention. The interpretation result acquiring unit corresponds to the interpretation result acquiring unit of the present invention. The biopsy result acquiring unit corresponds to the biopsy result acquiring unit of the present invention.

Basic Processing Flow

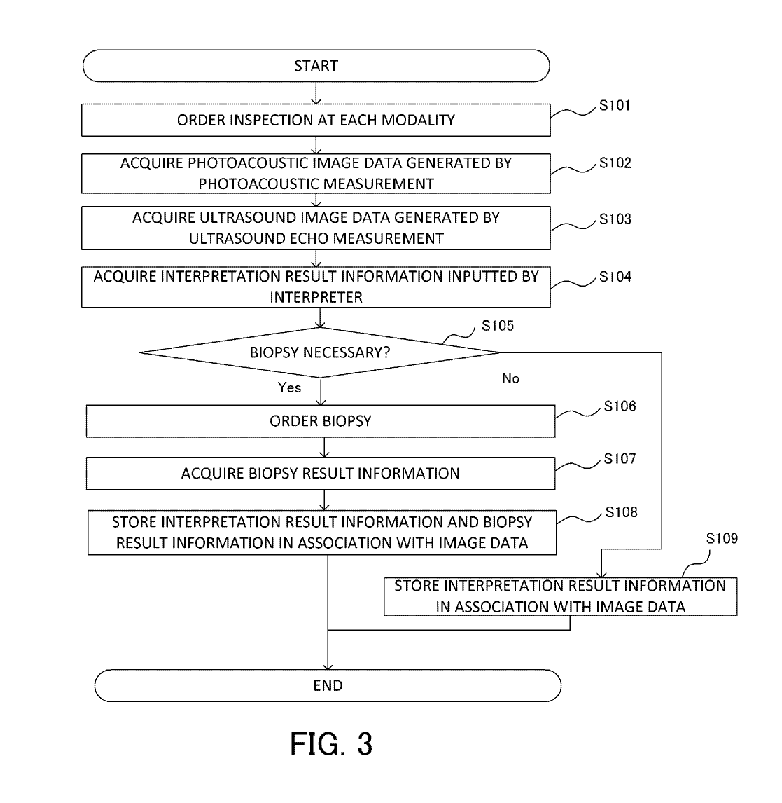

FIG. 3 is a flow chart depicting a basic processing flow.

In step S101, an examiner who determined that the image diagnosis of an examinee is necessary, orders an inspection with each modality using the ordering terminal 121 of the ordering system. A technician who received the order performs the photoacoustic measurement using the photoacoustic apparatus 131, and generates the photoacoustic image data 231. Thereafter the technician performs the ultrasound echo measurement using the ultrasound apparatus 132, and generates the ultrasound image data 232.

In steps S102 and S103, the measurement result acquiring unit 103 of the server 101 acquires the photoacoustic image data 231 and the ultrasound image data 232. The acquired image data is stored in the database 111. At this time, the image data processing unit 106 may process the data.

When the image data is acquired, interpretation is requested to the interpreter. The interpreter interprets the image, which is displayed on the interpreter terminal 141, based on the image data 241, and inputs a comment. For the image data 241, the photoacoustic image data 231 and the ultrasound image data 232 may be used as is, or may be used after performing the correction processing and the like.

In Example 1, the interpreter first refers to the ultrasound image, and specifies whether there is a region that may have a tumor inside the object (e.g. breast), and if a tumor exists specifies the position of the region. Then the interpreter refers to the photoacoustic image, and interprets the blood vessels in the specified region and the peripheral region thereof, generates blood vessel information, and includes this information in the interpretation result information. The blood vessel information is: the number of blood vessels existing around the tumor, a blood vessel density inside the tumor, oxygen saturation degree and the like, which is stored in the database as a part of the interpretation result information. Generally many blood vessels exist around a malignant tumor. Further, the blood inside the tumor has low oxygen. This means that whether a tumor is benign or malignant can be estimated using the number of blood vessels, the blood vessel density inside the tumor, and the oxygen saturation degree inside the tumor. If there is other useful information for diagnosis, this information may be included in the interpretation result information. The BI-RADS (Breast Imaging Reporting and Data System) category, which is estimated based on the image, may also be included in the interpretation result information.

The interpreter may estimate whether the tumor is benign or malignant based on the photoacoustic image, and include this estimation in the interpretation result information. Alternatively, the interpreter may estimate only the number of blood vessels, blood vessel density and oxygen saturation degree, and based on this data, the interpretation support software installed on the server 101 may estimate whether the tumor is benign or malignant. In other words, the interpretation support software causes the server to function as the interpretation support unit.

Then in step S104, the interpretation result acquiring unit 104 of the server 101 acquires the interpretation result information 242.

The server 101 presents the interpretation result information 242 to a physician using a presentation device (e.g. display). The physician determines whether biopsy is necessary based on comments and the like of the interpreter included in the interpretation result, and inputs the determination result to the server 101 using the input device (e.g. mouse, keyboard). Then in step S105, the determination result acquiring unit 107 of the server 101 acquires the inputted determination result, and determines the subsequent processing operations.

If a biopsy is necessary (YES in S105), processing advances to step S106, and an order for biopsy is outputted to the biopsy specialist. In the biopsy necessity determination by the physician, if the interpretation support software supports the biopsy necessity determination, the determination result acquiring unit 107 receives the interpretation result from the interpretation support software. It is also possible that the server 101 determines the necessity of a biopsy based on the interpretation result information 242, and orders the biopsy to the biopsy specialist. Further, it is also possible that the interpretation software installed on the server 101 performs the interpretation.

If biopsy is unnecessary (NO in S105), on the other hand, a follow up examination is performed. Processing advances to step S109, and the interpretation result information 242 and the image data with each modality are associated and stored in the database 111.

The biopsy specialist who received the order performs biopsy, acquires the determination result, and inputs this result to the biopsy specialist terminal. In step S107, the biopsy result acquiring unit 105 of the server 101 acquires the biopsy result information 251. In step S108, the server 101 associates the interpretation result information 242, the biopsy result information 251 and the image data with each modality, and stores this data in the database 111. The method of associating the data is arbitrary. Each data may be stored in separate tables and associated by keywords, or data tables may be integrated. Information to indicate whether the estimation of a benign/malignant tumor by the interpreter or the interpretation support software is correct or not may also be stored.

In the table for storing the photoacoustic image data or the ultrasound image data, or in the table for storing the interpretation result information, information to indicate whether or not there is a biopsy result corresponding to this image data or the interpretation (biopsy result flag) may be added. The existence of the interpretation result information or the biopsy result may be stored in an electronic clinical record of the examinee.

By the above flow, a useful database integrating the knowledge of the interpreter and the biopsy specialist can be constructed. In the database at least image data, interpretation result information and biopsy result information (if a biopsy is performed) are associated and stored. The patient information, interpreter information and image information may also be associated and stored.

By comparing the characteristic of the image with the interpretation result and the biopsy result based on the database constructed according to Example 1, the interpretation technique of photoacoustic tomography, which is a relatively new modality, can be improved. In particular, the data on the biopsy result can be used for determining a correct/incorrect comment of the interpreter and analyzing the skill, habit and tendency of the interpreter by corresponding the interpretation result and the biopsy result. Further, this data on biopsy result can also be used for training the interpreter based on this tendency. Furthermore, this data on biopsy result may be used for developing the interpretation software and the interpretation support software based on machine learning, which uses the image, interpretation result and biopsy result (definitive diagnosis) as teaching data.

### Modification

In Example 1, as the information that is associated with the image data and the interpretation result, and stored in the database, only the above mentioned biopsy result flag may be used instead of the biopsy result information alone. By referring to the biopsy result flag in the database, the physician can know whether the biopsy result exists, and can make a future diagnostic plan.

### Example 2

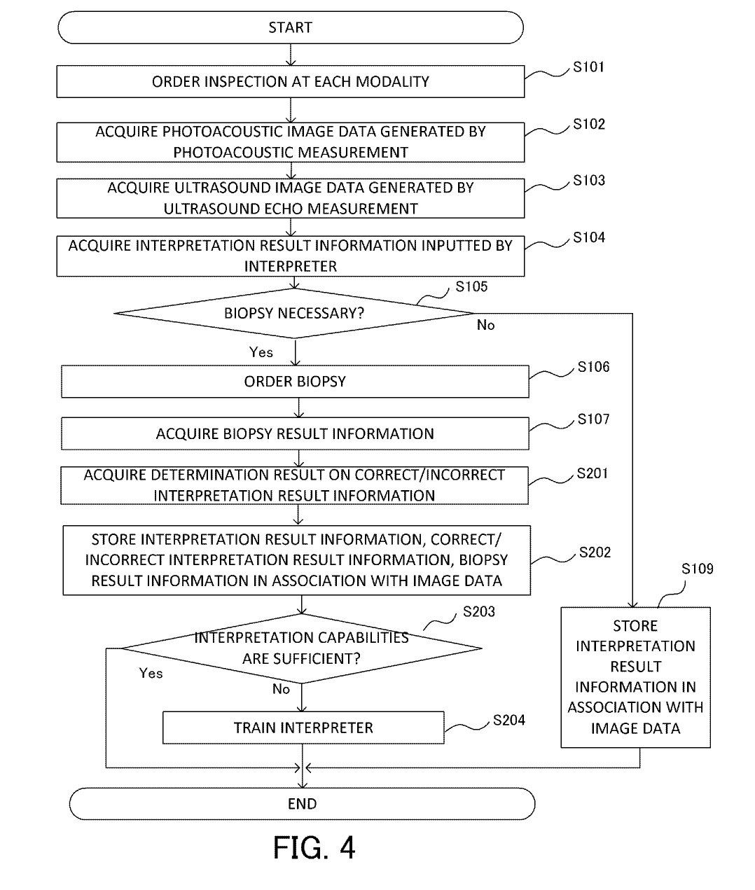

In Example 2, a case of using the database for training an interpreter will be described. The configuration of the information processing apparatus 1 is the same as Example 1. FIG. 4 is a flow chart of Example 2. Processing the same as FIG. 3 is denoted with the same reference sign, for which description is omitted. By operating in accordance with a program, the server 101 of Example 2 can function as a determining unit which determines a correct/incorrect interpretation, and as an interpreter evaluating unit which evaluates and examines the capabilities of the interpreter, and determine and execute training content.

Steps S101˜S107 and S109 are the same as Example 1.

After biopsy is performed based on the comment of the interpreter included in the interpretation result information, the correct/incorrect interpretation result information is determined in step S201 based on the definitive diagnosis result acquired by biopsy. Here, the determining unit of the server 101 may determine correct/incorrect by collating the biopsy result information and the interpretation result information. A supervisor or the like may determine correct/incorrect, and input the result to the server 101. The correct/incorrect may be determined for the information on the presence of a tumor and information whether it is benign or malignant. As long as the information is acquired by collating the interpretation result information and the biopsy result information, the interpreter evaluating unit may evaluate correct/incorrect interpretation result information.

In step S202, in addition to step S108 in FIG. 3, the determination result on correct/incorrect interpretation result information is associated with the other data and stored. The association target data varies, and not only the interpreter information and the interpretation result information, but also the patient information, the biopsy result information and the image information may be associated. The association method also varies, and cross referencing by keywords may be used, or a plurality of data may be integrated.

In step S203, the interpreter evaluating unit determines whether the capability of the interpreter is sufficient. The interpreter evaluating unit refers to the database, and calculates the correct answer rate of an interpreter based on the interpretation result information of the interpreter in the past, and correct/incorrect of the interpretation. The correct answer rate may be calculated based on all the interpretations in the past, or based on interpretations of a predetermined number of recent cases (e.g. 50 cases).

Processing ends if it is determined that the capability of the interpreter is sufficient (YES in S203).

If the correct answer rate of the interpreter is less than a predetermined standard (NO in S203), on the other hand, then training is provided to the interpreter. In this case, a training mode to perform training is executed. An example of training mode is the interpreter evaluating unit displaying an ultrasound image, a photoacoustic image and a biopsy result on the screen of the display device (display unit) to assist understanding for the interpreter. At this time, a photoacoustic image of a benign tumor and a photoacoustic image of a malignant tumor may be displayed as well. Any method that is helpful for training can be used.

As described above, if the database constructed according to the present invention is used, the interpretation capability of each interpreter can be evaluated. Furthermore, training can be provided to the interpreter when necessary, which improves the interpretation capability of the interpreter.

### Example 3

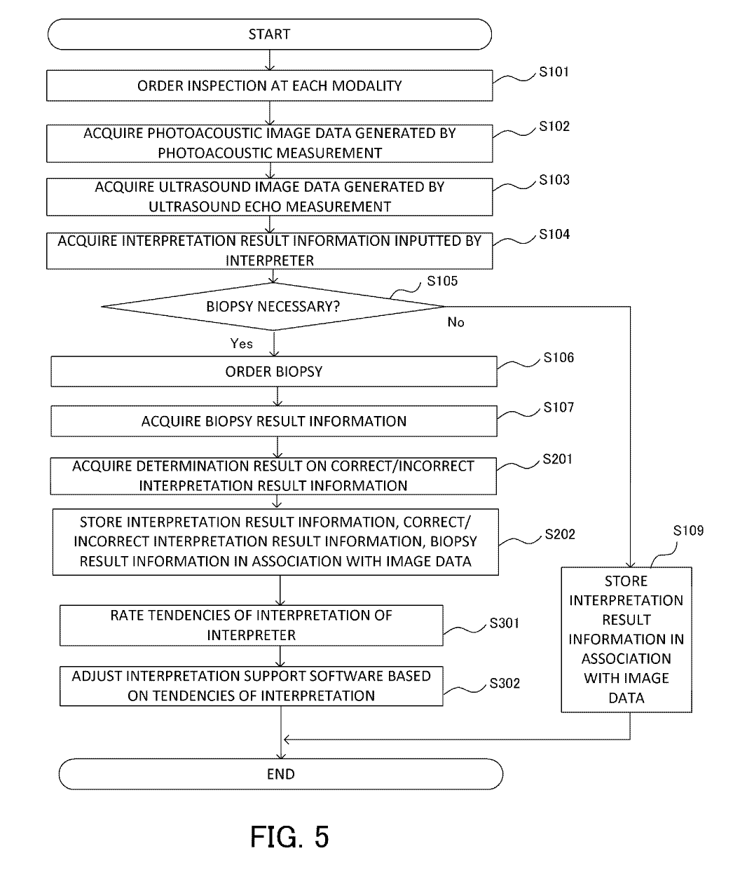

In Example 3, a case of using the database for recognizing the interpretation tendency of an interpreter will be described. The configuration of the information processing apparatus 1 is the same as Example 1. FIG. 5 is a flow chart of Example 3. Processing the same as FIG. 3 and FIG. 4 is denoted with a same reference sign, for which description is omitted. By operating in accordance with a program, the server 101 of Example 3 can function as a determining unit which determines correct/incorrect interpretation, and a tendency evaluating unit which evaluates the tendency of the interpretation of the interpreter, and adjusts the interpretation support software.

Steps S101˜S107 and S109 are the same as Example 1. Steps S301 and S302 are the same as Example 2. In step S104 of Example 3, the interpretation support software causes the server to function as the interpretation support unit. The interpretation support software estimates whether a tumor is benign or malignant based on the number of blood vessels included in the comments of the interpreter.

As described above, new blood vessels are generated around a tumor, and it is more probable that the tumor is malignant as the number of blood vessels is higher. Therefore the interpretation support software of Example 3 estimates that a malignant tumor exists when the number of blood vessels counted by the interpreter is a predetermined threshold or more. The threshold here is a value determined in advance based on general information. However, the number of blood vessels is counted by subjective estimation, and therefore the result differs depending on the interpreter. For example, if a certain interpreter has a tendency to overestimate when counting the number of blood vessels, the interpreter may determine in many cases that a benign tumor is a malignant tumor. Since biopsy exerts a huge stress on the body of a patient, preventing an unnecessary biopsy is desired.

Therefore in Example 3, in step S301, the tendency evaluating unit evaluates the tendency of interpretation of the interpreter. The tendency evaluating unit refers to the interpretation result information, the biopsy result information and the determination result of correct/incorrect interpretation, which are stored in the database, and evaluates the tendency of the interpretation. For example, of the cases when a tumor was determined as malignant based on the count of the blood vessels included in the interpretation result information and biopsy was performed, the ratio of cases when the tumor was actually benign is determined, and if this ratio is high, this means that this interpreter has tendency to overestimate when counting the number of blood vessels. If the number of blood vessels is estimated by an inspection with another modality or from previous inspection, the tendency of the interpretation can be evaluated using at least the data associating the information concerning the interpreter and the interpretation result information.

In step S302, the tendency evaluating unit adjusts the parameters of the interpretation support software based on the tendency of the interpretations of the interpreter. For example, if the interpreter has a tendency to overestimate when counting the number of blood vessels, the threshold of the number of blood vessels to estimate whether a tumor is benign or malignant is set to the higher side.

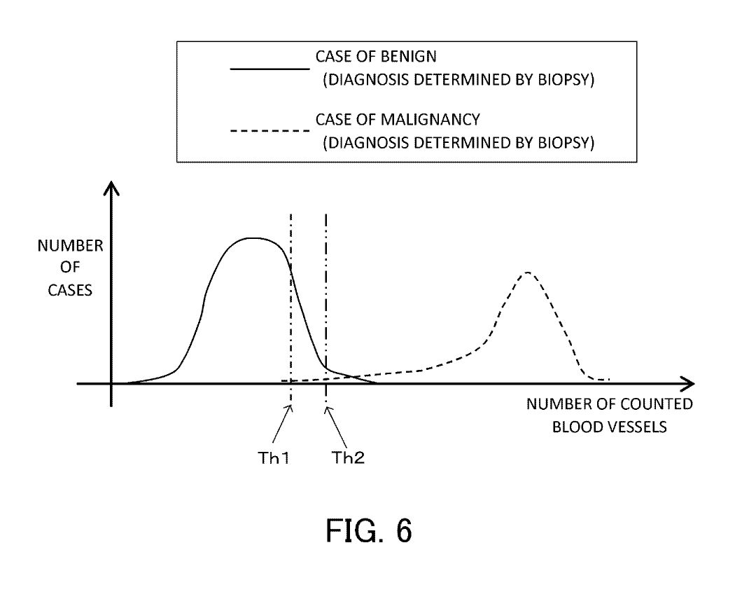

Embodiment 3 will be described in more detail with reference to FIG. 6. FIG. 6 is a graph created using a quantity of data (e.g. most recent 50 cases) of an interpreter stored in a database. Each entry of the acquired data includes at least information on “the number of blood vessels counted by this interpreter” and “whether the result of definitive diagnosis acquired by biopsy is benign or malignant”. In FIG. 6, benign cases and malignant cases are plotted respectively on a graph, of which the abscissa is the number of blood vessels, and the ordinate is a number of cases. The solid line indicates the cases determined as benign, and the broken line indicates the cases determined as malignant. The dashed line indicates a threshold Th1 that is initially set, and this threshold Th1 is used to determine whether biopsy is performed. This means that biopsy is performed if the number of counted blood vessels is Th1 or more, and biopsy is not performed if less than Th1.

According to FIG. 6, most cases were diagnosed as benign tumors, even if the number of counted blood vessels is Th1 or more. This means that many unnecessary biopsies were performed. Therefore, in Example 3, the threshold is changed from Th1 to Th2, indicated by a two-dot chain line in step S302. The threshold may be changed after data of a certain number of cases is acquired, or may be changed for each case.

The method of setting the number of blood vessels varies. For example, the threshold may be lowered for an interpreter who underestimates when counting blood vessels. Further, a lower limit may be set for the threshold based on the number of blood vessels which normally exist in a segment of an object. Besides the number of blood vessels, Example 3 may be applied to various values, such as blood vessel density and an oxygen saturation degree.

According to Example 3, the tendency of an interpretation can be recognized for each interpreter based on the cases, and the sensitivity of the interpreter to estimate whether the tumor benign or malignant can be appropriately adjusted, therefore unnecessary biopsies can be decreased without misdiagnosing a malignant tumor.

### Example 4

In Example 4, a method of training an interpreter based on the interpretation result information performed by a plurality of interpreters will be described. First, a plurality of (e.g. five) interpreters independently interpret photoacoustic images for a same case. When the interpretation result information of these five interpreters are stored in a database, the server 101 compares these interpreters. The comparison may be performed for each case, or may be performed after a certain amount of data is collected.

For example, if an average number of blood vessels counted by an interpreter is considerably different from the averages counted by the other 4 interpreters, this interpreter is recommended to undergo interpretation retraining.

Further, for each interpreter, correct/incorrect of a benign/malignant estimation result may be analyzed, so as to detect the cases in which the skill of each interpreter is strong or weak. For example, if the correct answer rate of an interpreter is low with respect to a relatively large tumor, this interpreter is recommended for training especially designed for such cases.

By evaluating the interpretation capability for each interpreter stored in a database like this, the interpretation capabilities of all interpreters can always be maintained at a high level.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™, a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-178749, filed on Sep. 19, 2017, which is hereby incorporated by reference herein in its entirety.

What is claimed is:

1. An information processing apparatus, comprising:
an image acquiring unit configured to acquire a photoacoustic image which originates from a photoacoustic wave generated by irradiating an object with light;
an interpretation result acquiring unit configured to acquire interpretation result information, which represents a result of interpreting the photoacoustic image;
a biopsy result acquiring unit configured to acquire biopsy result information, which represents a result of biopsy performed on the object; and
a data generating unit configured to generate data in which the photoacoustic image, the interpretation result information and the biopsy result information are associated with one another.

2. The information processing apparatus according to claim 1, wherein
the interpretation result information includes at least one of the number of blood vessels, a blood vessel density and an oxygen saturation degree of the object, and
the biopsy result information includes at least one of information on the existence of a tumor in the object, and information on whether the tumor is benign or malignant.

3. The information processing apparatus according to claim 1, wherein the data generating unit generates the data in which information indicating an interpreter who has performed the interpretation, the interpretation result information and the biopsy result information are associated with one another.

4. The information processing apparatus according to claim 1, further comprising an interpreter evaluating unit configured to evaluate an interpretation capability of the interpreter who has performed the interpretation, based on the data.

5. The information processing apparatus according to claim 4, wherein the interpreter evaluating unit evaluates the interpretation capability by comparing the interpretation result information and the biopsy result information.

6. The information processing apparatus according to claim 4, wherein the interpreter evaluating unit executes a training mode for interpretation in accordance with the result of evaluating the interpretation capability.

7. The information processing apparatus according to claim 4, wherein the interpreter evaluating unit evaluates the interpretation capability of the interpreter, based on the interpretation result information on a predetermined number of cases.

8. The information processing apparatus according to claim 1, further comprising a tendency evaluating unit configured to evaluate an interpretation tendency of the interpreter who has performed the interpretation.

9. The information processing apparatus according to claim 8, wherein
the data generating unit generates the data in which information indicating an interpreter who has performed the interpretation, the interpretation result information, and the biopsy result information are associated with one another, and
the tendency evaluating unit evaluates the interpretation tendency, based on the data.

10. The information processing apparatus according to claim 8, wherein the tendency evaluating unit evaluates the tendency when the interpreter counts the number of blood vessels, based on: the number of blood vessels included in the interpretation result information; information on whether a tumor is benign or malignant, which is estimated by the number of blood vessels; and the biopsy result information.

11. The information processing apparatus according to claim 8, further comprising an interpretation support unit configured to determine whether a tumor is benign or malignant, based on the interpretation result information, wherein
the tendency evaluating unit adjusts parameters of the interpretation support unit in accordance with the tendencies of the interpretation of the interpreter.

12. An information processing method, comprising:
an image acquiring step of acquiring a photoacoustic image which originates from a photoacoustic wave generated by irradiating an object with light;
an interpretation result acquiring step of acquiring interpretation result information, which represents a result of interpreting the photoacoustic image;
a biopsy result acquiring step of acquiring biopsy result information, which represents a result of biopsy performed on the object; and
a data generating step of generating data in which the photoacoustic image, the interpretation result information, and the biopsy result information are associated with one another.

13. A non-transitory computer readable storage medium storing a program causing a computer to execute an information processing method, comprising:
an image acquiring step of acquiring a photoacoustic image which originates from a photoacoustic wave generated by irradiating an object with light;
an interpretation result acquiring step of acquiring interpretation result information, which represents a result of interpreting the photoacoustic image;
a biopsy result acquiring step of acquiring biopsy result information, which represents a result of biopsy performed on the object; and
a data generating step of generating data in which the photoacoustic image, the interpretation result information, and the biopsy result information are associated with one another.
